(19) United States
(12) Patent Application Publication
(10) Pub. No.: US 2005/0096274 A1
(43) Pub. Date: May 5, 2005

(54) Bone morphogenetic protein and fibroblast growth factor compositions and methods for the induction of cardiogenesis

(76) Inventors: John W. Lough, Elm Grove, WI (US); Matthew R. Barron, Houston, TX (US)

Correspondence Address: QUARLES & BRADY LLP, 411 E. WISCONSIN AVENUE, SUITE 2040, MILWAUKEE, WI, 53202-4497, US

(21) Appl. No.: 10/951,396

(22) Filed: Sep. 28, 2004

Related U.S. Application Data
(62) Division of application No. 09/851,516, filed on May 8, 2001, now abandoned.
Application No. 09/851,516 is a continuation-in-part of application No. 09/056,513, filed on Apr. 7, 1998, now abandoned.

(51) Int. Cl. A61K 38/18

(52) U.S. Cl. 514/12; 424/93.7

(57) ABSTRACT

A composition comprising a purified mixture of a bone morphogenetic protein and a fibroblast growth factor is disclosed. In another embodiment, the present invention is a method of inducing cardiogenesis in cells of a non-cardiac lineage comprising the steps of exposing cells to the composition and observing the development of cardiac cells.

### CROSS-REFERENCE TO RELATED APPLICATIONS

This application is a divisional application of U.S. patent application Ser. No. 09/851,516, filed on May 8, 2001, which is a continuation-in-part application of U.S. patent application Ser. No. 09/056,513, entered into U.S. national stage on Apr. 7, 1998 based on the PCT application PCT/US97/14229, filed on Aug. 13, which claims the benefit of the U.S. provisional application 60/024,602, filed on Aug. 16, 1996. These applications are herein incorporated by reference.

### STATEMENT REGARDING FEDERALLY SPONSORED RESEARCH OR DEVELOPMENT

This invention was supported by NIH grant number HL39829. The U.S. Government may have certain rights to this invention.

### BACKGROUND OF THE INVENTION

Embryonic anterior lateral (AL) plate endoderm cells, which are necessary to support terminal cardiogenesis in stage 6 precardiac mesoderm, Sugi Y and Lough J, Dev Dyn 200:155-162 (1994), can also induce cardiogenesis in cells that are not in the cardiogenic pathway. Schultheiss T M et al., Development 121:4203-4214 (1995). To ascertain the molecular basis of these effects, many secretory products of AL endoderm have been identified. To date, these products include the vitamin A transport proteins, Barron M et al., Dev Dyn 212:413-422 (1998), as well as growth factors in the fibroblast growth factor (FGF) and transforming growth factor-β (TGF-β) families. FGFs 1, 2, alt-2 and 4, Parlow M H et al., Dev Biol 146:139-147 (1991); Zhu X et al., Dev Dyn 207:429-438 (1996), and activin-A, Kokan-Moore N P et al., Dev Biol 146:242-245 (1991), can mimic the ability of AL endoderm to support terminal cardiac differentiation in precardiac mesoderm. Sugi Y and Lough J, Dev Biol 169:567-574 (1995).

The art now lacks a composition capable of inducing cardiogenesis in non-precardiac cells.

### BRIEF SUMMARY OF THE INVENTION

The present invention is a composition comprising a purified mixture of a bone morphogenetic protein (BMP) and a fibroblast growth factor (FGF). Specifically, the BMP is from the group consisting of BMP-2, BMP-4, BMP-6, BMP-7, BMP-12 and other BMPs that can activate BMP receptor 1B. Preferably, the BMP is BMP-2 or BMP-4. The FGF is selected from the FGFs that can activate FGF receptor 1c, 2c, 3c and 4Δ. Preferably, the FGF is FGF-2 or FGF-4.

In another embodiment, the present invention is a method for inducing cardiogenesis in cells of non-cardiac lineage comprising the steps of exposing cells to a purified mixture of a BMP and a FGF and observing the development of rhythmical contractile cells expressing sarcomeric α-actin. The exposure may be either in vitro or in vivo.

In one embodiment of the present invention, the protein mixture is applied exogenously to the cells. In another embodiment of the present invention, the cells are transformed with genetic constructs encoding a BMP and a FGF. The genetic constructs are then allowed to express the cardiogenetic proteins.

It is an important feature of the present invention that cardiac cells can be induced from non-precardiac cells.

Other objects, features and advantages of the present invention will become apparent after examination of the specification, drawing and claims.

### BRIEF DESCRIPTION OF THE SEVERAL VIEWS OF THE DRAWINGS

FIGS. 1A and B describe the incidence of cardiogenesis in non-precardiac mesoderm cells treated with BMP-2 and FGF-4. FIG. 1A diagrams the heart-forming region (pre-cardiac tissue) and the non-precardiac tissue region of a stage 6 avian embryo. FIG. 1B is a graph of percent contractile explants that are obtained, versus treatment with FGF-4, BMP-2, or FGF-4 and BMP-2 combined.

FIG. 2 shows the relative ability of BMP isoforms to induce non-precardiac mesoderm.

FIG. 3 shows that the cardiogenesis inductive effect of neither BMP nor FGF can be replaced by activin-A, insulin or FGF-7. The number above each bar indicates the total number of explants that were evaluated.

FIG. 4 shows the dose-dependent induction of cardiogenesis by BMP-2. FIG. 5 shows incidence of cardiogenesis in explants treated with BMP or FGF for defined intervals of the cultured period.

### DETAILED DESCRIPTION OF THE INVENTION

The present invention is a composition and method for the induction of cardiogenesis in non-precardiac cells, preferably human cells. By “cardiogenesis” we mean the development of rhythmically and synchronously contractile cells that express sarcomeric α-actin from cells that are not part of the cardiac lineage.

Preferably, a cell can be identified as a cardiac cell by visual observation via microscopy. The Examples below demonstrate that a monoclonal antibody that recognizes sarcomeric α-actin can confirm that cells are expressing α-actin. The Examples below also demonstrate an alternative way of identifying a cell induced for cardiogenesis: an induction of expression of cardiac gene transcription factors such as SRF and cNkx-2.5 in the cell.

By “non-precardiac cells,” we mean cells that are capable of differentiation but not part of the cardiac lineage. For example, the following cells are non-precardiac: cells that will become fibroblasts, which make connective tissue, and cells that will become other mesoderm derivatives, such as skeletal muscle.

The composition that can induce cardiogenesis in non-precardiac cells comprises a purified mixture of a bone morphogenetic protein (BMP) and a fibroblast growth factor (FGF). By “purified” we mean that the proteins in question have been purified from native or recombinant bacterial sources. For example, a crude cell extract is “purified” as is a combination of proteins that have been individually purified to almost 100% homogeneity. In the Examples described below, the compositions that have been shown to induce cardiogenesis in non-precardiac cells include FGF-4 with BMP-2, and FGF-2 with BMP-2, BMP-4, BMP-7, BMP-6 or BMP-12. FGF-4 and FGF-2 have been shown to have indistinguishable cardiogenic efficacy. It is anticipated that FGF-4 can also work with BMP-4, BMP-7, BMP-6 or BMP-12 to induce cardiogenesis. FGFs exert their biological function through FGF receptors. FGF-2 and FGF-4 can activate FGF receptors 1c, 2c, 3c and 4A. It is anticipated that other FGFs that can activate these receptors, such as FGF-1, FGF-5, FGF-6, FGF-8 and FGF-9, can also work with BMPs to induce cardiogenesis. For example, FGF-8, which can activate FGF receptors 2c, 3c and 4A, have been shown to be involved in zebrafish heart induction and development. Feifers, F et al., Development 127:225-235 (2000). FGF-7 cannot activate any of the above FGF receptors and has been shown to lack the ability to induce cardiogenesis with BMPs.

Information regarding FGFs and their receptors are reviewed in the following articles, all of which are hereby incorporated by reference: Ornitz, D M and Itoh, N, Genome Biol. 2(3): reviews 3005 (2001); Szebenyi, G et al., Int. Rev. Cytol. 185: 45-106 (1999); Ornitz, D M et al., J. Biol. Chem. 271(25): 15292-15297 (1996).

BMPs also exert their biological function through receptors. BMP-2 and BMP-4 can activate BMP receptors that contain the 1B subunit. It is anticipated that other BMPs that can activate BMP receptors that contain the 1B subunit can also work with FGFs to induce cardiogenesis. The preferred composition for cardiogenesis is one of a BMP selected from BMP-2 and BMP-4, and a FGF selected from FGF-2 and FGF-4 since such a composition gives high cardiogenic efficacy.

BMP-2, BMP-4, BMP-6 and BMP-7 are disclosed, for instance, in U.S. Pat. Nos. 5,168,050, 5,116,738, 5,106,748 and 5,141,905. BMP-12 is disclosed in PCT application WO 95/16035. The disclosures of all of the above-identified application and patents are hereby incorporated by reference.

A typical source for BMP-2 is bone or recombinant human BMP-2 that is expressed in bacteria. Preferred sources for FGF-2, FGF-4 and BMP-4 are from recombinant bacteria that express the human proteins. BMPs can be purchased from Genetics Institute (Cambridge, Mass.). Some specific BMPs such as BMP-2, BMP-4, BMP-6 and BMP-7 can also be purchased from R&D Systems (Minneapolis, Minn.). FGFs including FGF-1, FGF-2, FGF-4, FGF-5, FGF-6, FGF-8 and FGF-9 can be purchased from R&D Systems (Minneapolis, Minn.).

The compositions of the invention may comprise, in addition to a BMP and an FGF protein, other therapeutically useful agents, including growth factors such as epidermal growth factor (EGF), transforming growth factor (TGF-α and TGF-β), activins, inhibins, and insulin-like growth factor (IGF).

The compositions of the present invention may also include an appropriate matrix. For instance, one might desire a matrix for supporting the composition and providing a surface for growth of cardiomyocytes and/or other tissue growth. The matrix may provide slow release of the protein and/or the appropriate environment for presentation thereof and an appropriate environment for cellular infiltration. Such matrices may be formed of materials presently in use for other implanted medical applications.

The choice of matrix material is based on biocompatibility, biodegradability, mechanical properties, cosmetic appearance and interface properties. The particular application of the compositions will define the appropriate formulation. Potential matrices for the compositions may be biodegradable and chemically defined polymers, such as polymers of polylactic acid, polyglycolic polyorthoesters and polyanhydrides. Other potential materials are biodegradable and biologically well defined, such as collagen. Further matrices comprise pure proteins or extracellular matrix components. Other potential matrices are nonbiodegradable and chemically defined, such as sintered hydroxyapatite, bioglass, aluminates, or other ceramics. Matrices may comprise combinations of any of the above mentioned materials and other suitable types of material and may be altered in composition and processing to alter pore size, particle size, particle shape, and biodegradability.

In view of the Examples described below, the concentration of both the BMP and the FGF used to induce cardiogenesis can range from about 5 ng/ml to about 1,000 ng/ml. Preferably, the BMP concentration and the FGF concentration are each about 50 ng/ml. The BMP and the FGF will be mixed in a 1:1 molar ratio, preferably.

By “1:1” we mean a variation of at least 20% is still permissible. However, other ratios may result in cardiogenesis and may also be suitable.

In the Examples described below, short initial treatment with a BMP and a FGF is effective in inducing cardiogenesis in a portion of the non-precardiac cells. Longer treatment with both BMP and FGF can induce cardiogenesis in a larger portion of the non-precardiac cells. For maximum effect, an initial treatment with both BMP and FGF followed by a continued treatment solely with BMP is required.

One way to treat cells with a mixture of a BMP and a FGF is to exogenously apply the mixture to the cells. Another way to treat cells with a mixture of a BMP and a FGF is to transfect DNA sequences encoding the proteins of the BMP and the FGF into the cells and then make the cells produce the BMP and FGF proteins.

Potential uses of the compositions of the present invention include use of the composition to treat patients with cardiac tissue damage or stress. For example, as an adjunct to surgical procedures, cultured cells which are capable of differentiation into cells of cardio- or cardiomyocyte lineage are implanted into the damaged or stressed tissue and the composition may be applied directly to damaged or stressed tissue. Cells that may be useful in this and other applications of the present invention include non-precardiac embryo mesoderm cells, stem cells (See, e.g., U.S. Pat. No. 6,200,806), and other types of non-terminally differentiated cells that are susceptible to the induction of cardiogenesis.

Alternatively, the composition may be used to treat cells, whether autologous or heterologous, to promote the growth, proliferation, differentiation and/or maintenance of cells of a cardio- or cardiomyocyte lineage. The cells thus treated may then be applied to the damaged or stressed tissue, either alone or in conjunction with the protein composition of the present invention.

In another embodiment, DNA sequences encoding the proteins of the present compositions may be transfected into cells, rendering the cells capable of producing the BMP and FGF proteins. The transfected cells, which are capable of producing the BMP and FGF proteins, may then be implanted at the site of damaged or stressed tissue.

An appropriate matrix may be used with any of the above embodiments in order to maintain the composition and/or cells at the site of damaged or stressed tissue. Alternatively, an injectable formulation of the composition may be used for administration of the compositions of protein and/or cells. The above may also be used for prophylactic measure in order to prevent or reduce damage or stress to tissue.

The dosage regimen for a particular application will be determined by the attending physician considering various factors which modify the action of the protein composition, e.g. amount of tissue desired to be formed, the site of tissue damage, the condition of the damaged tissue, the size of a wound, type of damaged tissue, the patient's age, sex, and diet, the severity of any infection, time of administration and other clinical factors. The dosage may vary with the type of non-precardiac cells used, the type of matrix used in the reconstitution and the types of proteins in the composition. The addition of other known growth factors, such as IGF I (insulin like growth factor I), to the final composition, may also affect the dosage.

### EXAMPLE 1

In General

Because immunostaining to detect additional TGF-β family growth factors in HFR endoderm revealed a provocative expression pattern for Drosophila decapentaplegic (dpp)-like proteins, we performed a degenerate reverse transcription/polymerase chain reaction (RT/PCR) screen to identify vertebrate dpp-like factors that are expressed by these cells. Among more than 50 PCR products sequenced to date, over half are identical to bone morphogenetic factor-2 (BMP-2).

We then investigated whether BMP-2 mimics the cardiogenic effects of HFR endoderm on precardiac mesoderm, as well as its ability to re-specify non-precardiac mesoderm to the cardiac lineage. We report here that, when present as the only supplement in defined medium, BMP-2 cannot support viability of either precardiac or non-precardiac mesoderm. Although FGF-4 can support cardiogenesis in precardiac mesoderm, this factor did not induce cardiogenesis in non-precardiac mesoderm, although explant growth was maintained. Remarkably, however, treatment of non-precardiac mesoderm with combined FGF-4 and BMP-2 induced cardiogenesis in a high incidence of explants, indicating that this combination of growth factors is able to re-specify embryonic cells to the cardiac lineage.

Materials and Methods

Explantation and Culture of Embryonic Mesoderm: Chicken embryos were staged according to the criteria of Hamburger and Hamilton. Hamburger V and H L Hamilton, J Morphol 38:49-92 (1951). Anterior lateral plate precardiac mesoderm, and non-precardiac mesoderm from the posterior half of stage 6 embryos, was micro-dissected, explanted to Lab-Tek chamber slides and cultured in M199 as previously described. Sugi Y and J Lough, Dev Dyn 200:155-162 (1994); Sugi Y and J Lough, Dev Biol 169:567-574 (1995). Growth factors were added to the indicated final concentrations after explants attached to the fibronectin substrate. Human recombinant FGF-4 was purchased from R&D Systems. Human recombinant BMP-2 was provided by the Genetics Institute (Cambridge, Mass.). Medium, including growth factors, was changed daily.

Immunohistochemistry: Biochemical differentiation was monitored by immunohistochemistry, using a monoclonal antibody that recognizes sarcomeric α-actin (Sigma, Cat. No. A-2172); the secondary antibody was fluorescent isothiocyanate (FITC)-labeled goat anti-mouse IgM (Cappel). Decapentaplegic-like protein was localized using a polyclonal antibody (1:1,000) provided by Dr. F. Michael Hoffmann (University of Wisconsin), Panganiban, G. E. F. et al., Mol. Cell. Biol. 10:2669-2677 (1990), that recognizes Drosophila decapentaplegic. Controls consisted of identically diluted normal rabbit serum which was used as the primary antibody, and omission of the primary antibody. The secondary antibody was FITC-conjugated goat anti-rabbit IgG (1:500). All immunohistochemical procedures, including determinations of 5′-bromodeoxyuridine incorporation, have been previously described. Sugi Y and J Lough, Dev Biol 169:567-574 (1995).

Reverse Transcription-Polymerase Chain Reaction (RT/PCR): RNA from microdissected stage 6 HFR endoderm was purified, with 5 μg linear polyacrylamide as carrier, using RNAstat (Tel-Test, Inc.). Complementary DNA was synthesized by M-MLV reverse transcriptase-mediated extension of oligo-dT-primed RNA. To ensure the absence of contaminating genomic DNA, non-reverse transcribed RNA was simultaneously processed. Degenerate primers were designed to recognize conserved domains in the TGF-β dpp subfamily. The upstream primer was 5′-TGGAATTCGGITGGVAIGAYTGGAT-3′ (96-fold degenerate) (SEQ ID NO:1); the reverse complement of the downstream target was 5′-GAGGATCCGGIACRCARCAIGCYTT-3′ (128-fold degenerate) (SEQ ID NO:2).

Complementary DNAs were amplified using Thermus aquaticus (Taq) DNA polymerase (Promega) with 40 cycles of denaturation (94° C., 1 minute), primer annealing (45° C., 1.5 minutes) and extension (72° C., 2 minutes). Complementary DNAs in the predicted 200 bp product were cloned into pCRScript (Stratagene). Identity of cloned inserts was determined by sequencing and comparison with the GenBank/EMBO database.

Results

Immunohistochemical Localization of DPP-Like Protein in HFR Endoderm: To ascertain whether Drosophila decapentaplegic (dpp)-like proteins were associated with HFR endoderm, the anti-dpp immunostaining pattern of cultured HFR endoderm was determined in comparison with explanted precardiac mesoderm. The periphery of HFR endoderm cells exhibited intense staining, which was not observed in precardiac mesoderm or in control explants stained with normal rabbit serum.

RT/PCR Demonstration of BMP-2 in HFR Endoderm: Because dpp is 75% homologous with BMPs 2 and 4, it was considered that the antigens described above represented these factors or perhaps other members of the TGF-β dpp subgroup. To identify dpp mRNAs that are expressed by HFR endoderm, a RT/PCR screen was performed using degenerate primers targeted to conserved domains in this subgroup. A single 200 bp PCR product, which was the predicted size for dpp cDNAs, was cloned and sequenced to identify individual dpp-like factors that are expressed by HFR endoderm. Among approximately 50 cDNAs sequenced to date, more than half were identical to BMP-2 over a 162 base stretch corresponding to nucleotides 800-962 of the chicken homologue, Francis, P. H. et al., Development 120:209-218 (1994), a domain specified by the primers. No other known or novel member of the dpp subgroup has been identified. Although the remaining, cloned cDNAs exhibited sequences that were similar to each other, these are not homologous to any database entries. These findings suggest that BMP-2 is the major member of the dpp group that is expressed by HFR endoderm.

BMP-2 & FGF-4 Induce Cardiogenesis in Non-Precardiac Mesoderm: Based on these results, it was of interest to determine whether BMP-2, when present alone in defined medium, could emulate the cardiogenic effect of HFR endoderm on precardiac mesoderm. Unlike FGFs, Sugi Y and J Lough, Dev Biol 169:567-574 (1995); Zhu X et al., Dev Dyn 207:429-438 (1996), BMP-2 supported neither survival nor differentiation of precardiac mesoderm. However, as anticipated, inclusion of FGF-4 with BMP-2 triggered terminal cardiogenesis in precardiac mesoderm.

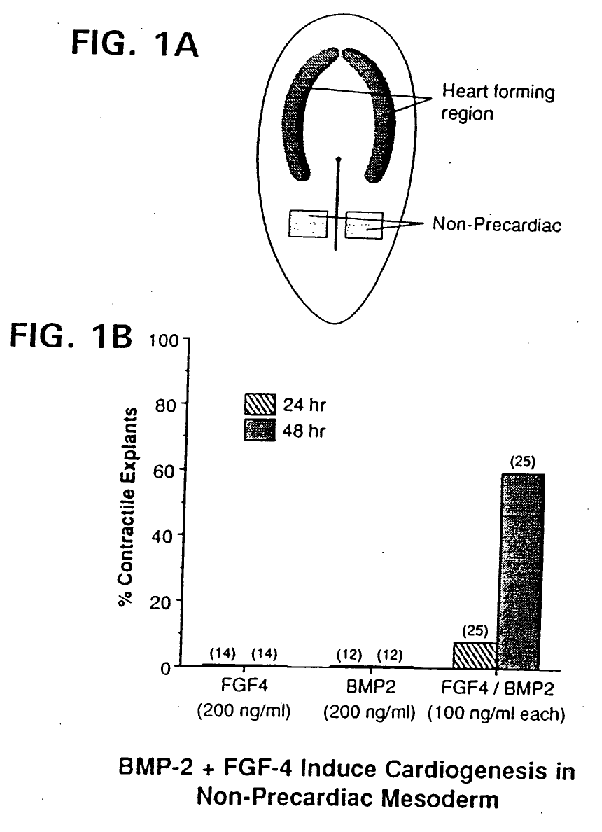

FIGS. 1A and B describe the incidence of cardiogenesis in non-precardiac mesoderm treated with BMP-2 and FGF-4. Non-precardiac mesoderm was explanted from the posterior half of stage 6 embryos and cultured in the presence of FGF-4 and/or BMP-2 at the indicated concentrations. Whereas neither FGF-4 nor BMP-2 alone induced cardiogenic differentiation in any explant, the majority of explants treated with both growth factors exhibited cellular multilayering and rhythmic contractility within 1 or 2 days. Referring to FIG. 1B, numbers in parentheses indicate experimental repetitions that were conducted during the aggregate of five experiments, each of which included approximately five replicate explants; the incidence of differentiation (40-60%) was similar within each experimental repetition.

As diagramed in FIG. 1A, these determinations were performed on non-precardiac mesoderm explanted from the posterior region of stage 6 embryos. Cells in this area are destined to become extraembryonic mesoderm and lateral plate mesoderm that is not cardiogenic. Nicolet, G, Adv. Morphogenesis 9:231-262 (1971). As shown in FIG. 1B, neither FGF-4 nor BMP-2 alone could induce formation of contractile explants in non-precardiac mesoderm. Cells cultured with BMP-2 alone detached from the culture dish and did not survive; and, although treatment with FGF-4 alone supported cellular proliferation as evidenced by 5′-bromodeoxyuridine incorporation, differentiation was never observed. Remarkably however, the combined presence of FGF-4 and BMP-2 caused cardiogenic differentiation in over half of the non-precardiac mesoderm explants (FIG. 1B), as indicated by formation of a multicellular vesicle which exhibited rhythmic contractility and sarcomeric α-actin differentiation. Because differentiation of non-precardiac mesoderm was not usually observed until the second day in vitro (FIG. 1B), in contrast to precardiac mesoderm in which differentiation is observed on day one, the occurrence of a re-specification step in non-precardiac mesoderm explants is suggested. These findings indicate that these growth factors synergistically function to induce cardiogenesis in cells that are not fated to the cardiac lineage.

### EXAMPLE 2

Experimental Procedures

Explantation and Culture of Non-Precardiac Mesoderm: Chicken embryos judged according to the criteria of Hamburger and Hamilton, Hamburger V and H L Hamilton, J Morphol 38:49-92 (1951), to be at stage 6 were exclusively used in this study. Anterior lateral plate precardiac mesoderm, and non-precardiac mesoderm from the posterior lateral plate, were explanted and cultured as previously described. Lough J et al., Dev Biol 178:198-202 (1996). Growth factors were added to the indicated final concentrations after explants attached to the fibronectin substrate. Activin-A and human recombinant FGFs 2, 4, and 7 were purchased from R&D Systems (Minneapolis, Minn.). Insulin was purchased from Sigma Chemical Company (St. Louis, Mo.). Leukocyte inhibitory factor (LIF: murine, cat. no. 13275-029) was purchased from Gibco BRL, Gaithersburg, Md. The Genetics Institute (Cambridge, Mass.) generously contributed human recombinant BMPs 2, 4, 6, 7, 12, and 13. Medium, including growth factors, was changed daily except as otherwise noted.

Formation of cardiac muscle in explants was verified by the morphogenesis of multilayered vesicles containing cells whose contractions were always rhythmic and synchronous. At the biochemical level contractile explants expressed sarcomeric α-actin as detected by immunostaining, ventricular myosin heavy chain (VMHC), Stewart A F et al., J Mol Evol 33:357-366 (1991), as detected by RT/PCR and, in particular, Nkx-2.5, a transcription factor that is not expressed in skeletal muscle tissue. Explants that did not become multilayered neither contracted nor exhibited biochemical differentiation; these were scored as non-contractile. Multilayered explants that exhibited contractility, which was always rhythmic (indicative of cardiac myogenesis), were scored as contractile. The appropriateness of these assessments for cardiogenesis and the absence of skeletal muscle differentiation in these explants has previously been discussed. Sugi Y and J Lough, Dev Dyn 200:155-162 (1994); Yatskievych T A et al., Development 124:2561-2570 (1997). Statistical analysis of explant differentiation was performed by pairwise comparisons using a modified z-test with a pooled estimate of standard error.

Immunohistochemistry: Biochemical differentiation was monitored by immunohistochemistry as described previously, Sugi Y and J Lough, Dev Dyn 200:155-162 (1994), using a monoclonal antibody that recognizes sarcomeric α-actin (Sigma, St. Louis, Mo.; Cat. No. A-2172); the secondary antibody was fluorescent isothiocyanate (FITC)-labeled goat anti-mouse IgM (Organon Teknika (Cappel), Durham, N.C.).

Reverse Transcription/Polymerase Chain Reaction (RT/PCR): Determinations of gene expression in these explants, each of which contains only approximately 10,000 cells, required sensitivity provided by the reverse transcription/polymerase chain reaction (RT/PCR). RNA from individual non-precardiac mesoderm explants was purified, using 5 mg linear polyacrylamide as carrier, with RNAstat (Tel-Test, Friendswood, Tex.). Purified RNA was treated with DNase I (Boehringer Mannheim, Indianapolis, Ind.) to remove any contaminating genomic DNA. Reverse transcription (RT) was performed using oligo-dT as the primer and with M-MLV reverse transcriptase (Promega, Madison, Wis.). To ensure the absence of contaminating genomic DNA, samples containing RNA that was not reverse-transcribed were simultaneously processed. One-tenth of the resultant RT product was used as template for PCR reactions performed in a 25 ml reaction mixture containing 1.5 mM MgCl₂ that was catalyzed with Thermus aquaticus (Taq) DNA polymerase (Promega).

Standard PCR amplifications were performed using 35 cycles of denaturation (94° C., 30 sec), annealing (60° C., 60 sec), and extension (72° C., 120 sec). Two-fifths of each PCR product were separated on a 1.5% agarose gel and stained with ethidium bromide. PCR products were sized by comparing migration to that of standard base pair markers (100 bp ladder; Gibco BRL). Semi-quantitative PCR was performed by including 1.0 μCi α-³²P-dCTP in the reaction mixture and using only 28 cycles, during which accumulation of PCR products was linear and which amplified quantifiable amounts of Nkx-2.5 and SRF cDNAs without generating saturating amounts of GAPDH PCR product. Two-fifths of each PCR product were separated on a 4.5% acrylamide gel and bands were visualized on a Storm 860 Optical Scanner (Molecular Dynamics, Sunnyvale, Calif.). Amounts of PCR product relative to GAPDH were estimated by Image-Quant analysis. Size markers were provided by the 100 bp ladder (Pharmacia, Gaithersburg, Md.) which was end-labeled with α-³²P-dCTP using the Klenow reaction.

All oligodeoxynucleotide primers were purchased from Operon (Alameda, Calif.). The primer pair for glyc-eraldehyde-3-phosphate dehydrogenase (GAPDH) was 5′-ACGCCATCACTATCTTCCAG-39 (SEQ ID NO:3) (forward) and 5′-CAGCCTTCACTACCCTCTTG-3′ (SEQ ID NO:4) (reverse), designed to amplify a 579 bp PCR product corresponding base pairs 265-843 of chicken GAPDH. Panabieres F et al., Biochem Biophys Res Comm 118:767-773 (1984). The primer pair for Nkx-2.5 was 5′-CTACGAACTGGAGAGAAGGT-3′ (SEQ ID NO:5) (forward) and 5′-GTAG-GCGTTGTAGCTATAGG-3′ (SEQ ID NO:6) (reverse), designed to amplify a 295 bp PCR product corresponding to base pairs 471-765 of chicken Nkx-2.5. Schultheiss T M et al., Development 121:4203-4214 (1995). The primer pair for serum response factor (SRF) was 5′-CAGCAACTCTCTCGTACGGA-3′ (SEQ ID NO:7) (forward) and 5′-TCTGCAGGACAGCTCCAGGT-3′ (SEQ ID NO:8) (reverse), designed to amplify a 343 bp PCR product corresponding to base pairs 1183-1525 of chicken SRF. Croissant J et al., Dev Biol 177:250-264 (1996). The primer pair for GATA-4 was 5′-CTCCTACTCCAGCCCTTACC-3′ (SEQ ID NO:9) (forward) and 59-GCCCTGTGCCATCTCTCCTC-3′ (SEQ ID NO:10) (reverse), which amplifies a 224-bp segment of chicken GATA-4 (bp 300-523; Laverriere et al., J Biol Chem 269:23177-23184 (1994). The primer pair used to amplify Dlx-5 was 5′-GCTCCGCCGGCACCTACCC-3′ (SEQ ID NO:11) (forward) and 5′-GGAGCGCGACGAGCCCTGAG-3′ (SEQ ID NO: 12) (reverse), which amplifies a 452-bp segment of chicken Dlx-5 (bp 296-747). Ferrari D et al., Mech Dev 52:257-264 (1995). The primer pair used to amplify VMHC was 5′-GGCGACTCTTGATGAGAACA-3′ (SEQ ID NO:13) (forward) and 5′-GCTTCCAGCTCCTCTTCCAG-3′ (SEQ ID NO: 14) (reverse), generating a 425-bp PCR product corresponding to bp 255-680 in the sequence reported by Stewart A F et al., J Mol Evol 33:357-366 (1991).

Results

BMP and FGF Are Specific in Their Ability to Induce Cardiogenesis:

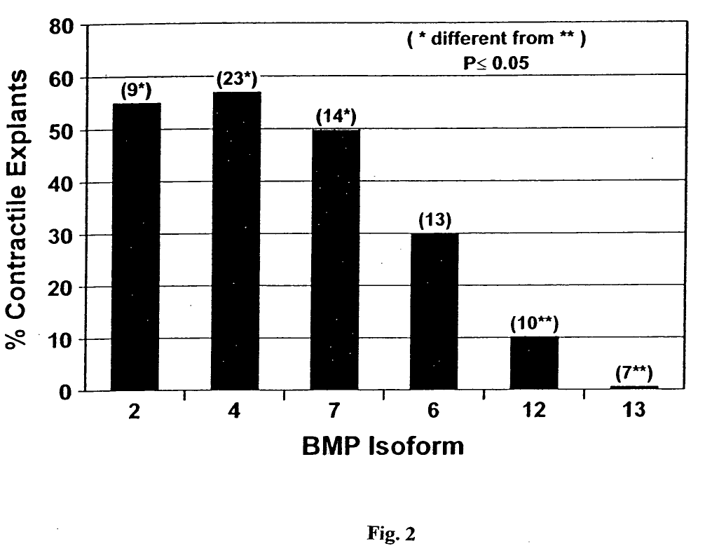

Determinations were performed to identify the most cardiogenic homologues of BMP and FGF. FGF-2 was used to test cardiogenic efficacies of different BMPs. Based on availability and biological activity in other systems, BMPs 2, 4, 6, 7, 12,and 13 were selected for evaluation. Non-precardiac mesoderm was explanted from the posterior lateral plate of stage 6 embryos as described in Experimental Procedures. Explants were exposed to a combination of 50 ng/ml FGF-2 and 50 ng/ml of each BMP isoform for 48 hours, after which cardiogenic differentiation was assessed from observations of rhythmic contractility. As shown in FIG. 2, it was observed that BMPs 2, 4,and 7 had similar activity, supporting cardiogenesis in 50-60% of explants. These factors were followed in potency by BMPs 6 and 12, which respectively generated cardiogenic vesicles in 30% and 10% of explants. BMP-13 was not cardiogenic. The number above each bar in FIG. 2 indicates the total number of explants that were evaluated. When BMP-2 was used to combine with FGF-2 or FGF-4 for cardiogenic efficacy evaluations, FGF-2 and FGF-4 showed similar cardiogenic efficacies.

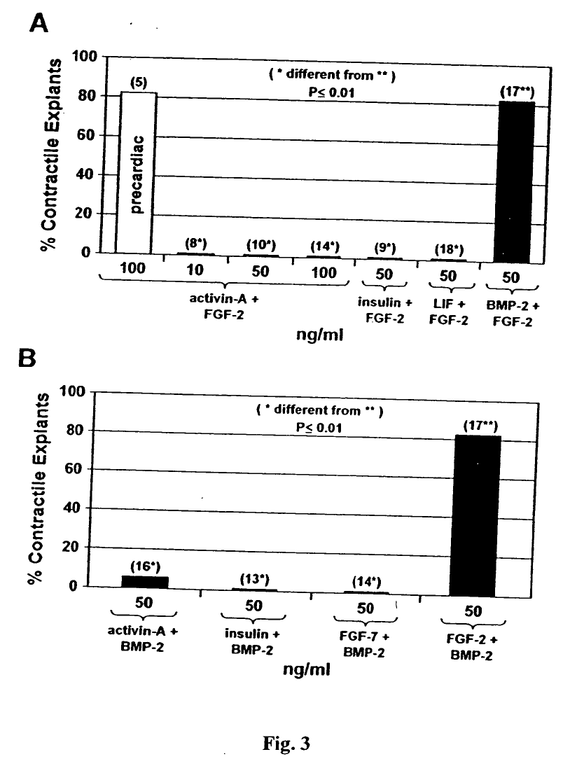

Activin-A, a related member of the TGFβ family, can mimic the ability of hypoblast to induce cardiac myogenesis in stage 1 epiblast, Yatskievych T A et al., Development 124:2561-2570 (1997), and can mimic the ability of stage 6 endoderm to support the completion of cardiogenesis in precardiac mesoderm, Sugi Y and J Lough, Dev Biol 169:567-574 (1995); insulin similarly mimics endoderm's effects. Sugi Y and J Lough, Dev Biol 169:567-574 (1995). Leukocyte inhibitory factor (LIF) is transduced via the cardiotrophin receptor to induce hypertrophy in differentiated cardiac myocytes. Sheng Z et al., Development 122:419-428 (1996). It was therefore of interest to ascertain whether activin-A, insulin, or LIF could replace the cardiogenic effect of BMP-2 on posterior non-precardiac mesoderm. Posterior non-precardiac mesoderm from stage 6 embryos was cultured in Medium 199 plus FGF-2 (50 ng/ml) plus either activin-A, insulin or BMP-2 at the indicated concentrations. As shown in FIG. 3A, explants in which BMP-2 was replaced with 10-100 ng/ml activin-A, 50 ng/ml insulin or LIF did not differentiate. (Evidence that the activin-A used in these determinations was bioactive was shown by its ability to support terminal differentiation in simultaneously prepared precardiac mesoderm explants.) Data indicated by the open bar at far left in FIG. 3A was a positive control to ensure the efficacy of activin-A, which when present alone (100 ng/ml) induced terminal cardiogenesis in precardiac mesoderm.

Because activin-A and insulin, like FGFs 1, 2, or 4, can mimic the ability of stage 6 endoderm to support terminal differentiation in precardiac mesoderm, Sugi Y and J Lough, Dev Biol 169:567-574 (1995); Zhu X et al., Dev Dyn 207:429-438 (1996), it was also of interest to ascertain whether activin-A or insulin could replace the cardiogenic effect of FGF-2 on posterior non-precardiac mesoderm. The experiments shown in FIG. 3B were similar to those in FIG. 3A, except that non-precardiac mesoderm explants were treated with 50 ng/ml BMP-2, plus either activin-A, insulin, FGF-7, or FGF-2 at the indicated concentrations. As shown in FIG. 3B, with the exception of one explant that differentiated in the presence of activin-A, none of these factors could replace FGF-2's cardiogenic effect. Because FGF-2 is highly homologous to FGF-4, which also has cardiogenic potency, it was of interest to determine whether a more distantly related FGF protein such as FGF-7 could induce cardiogenesis. As shown in FIG. 3B, FGF-7 could not induce cardiogenesis.

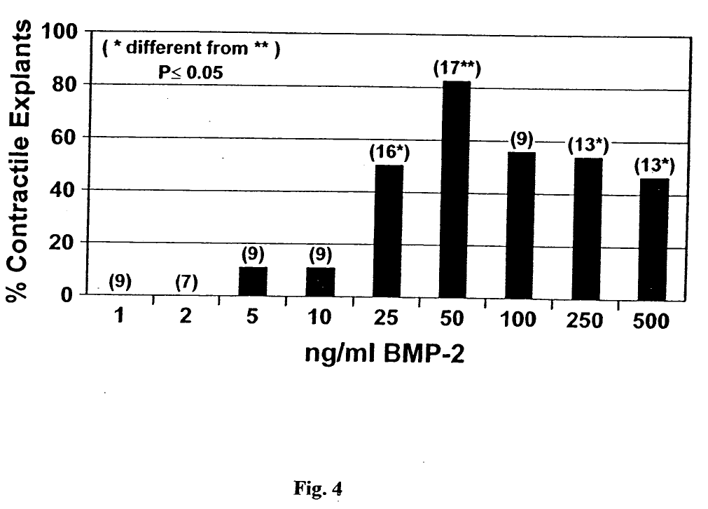

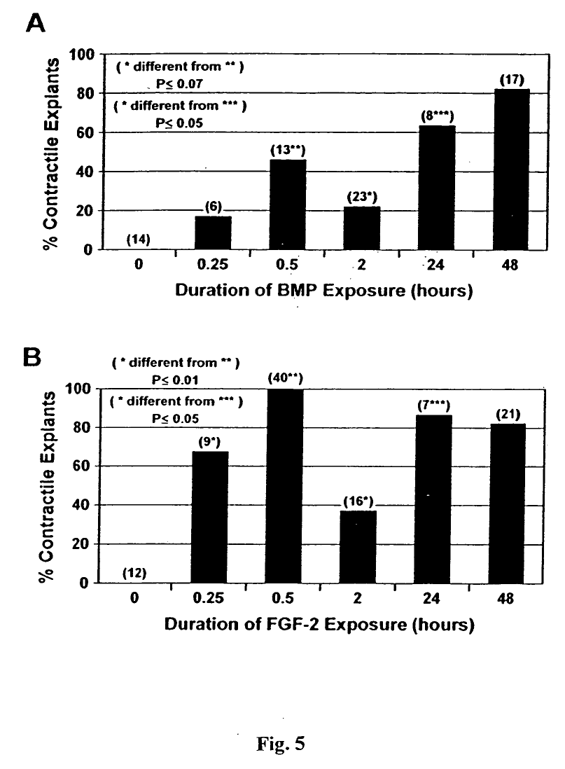

Optimal Concentrations of BMP-2 and FGF-2: Having determined that BMPs 2 and 4 were the most potent cardiogenic isoforms, it was decided to utilize BMP-2 for the remaining experiments. To assess the optimum concentration of BMP-2, explants were ex-posed to a range of 0-500 ng/ml, while maintaining FGF-2 at 50 ng/ml. Explants were evaluated for rhythmic contractility and sarcomeric α-actin immunostaining. As shown in FIG. 4 (the number above each bar indicates the total number of explants that were evaluated), concentrations lower than 5 ng/ml did not support cardiogenesis, while treatment with 5-10 ng/ml was minimally effective. Treatment with 25 ng/ml caused approximately 50% of the explants to differentiate, whereas maximal cardiac differentiation was obtained at a concentration of 50 ng/ml. When levels were increased to 100-500 ng/ml, the percentage of cardiogenic explants declined. These results indicate that 50 ng/ml, which incidentally was the concentration used to evaluate efficacy of the BMP isoforms in FIG. 2, was optimal for cardiogenesis. Similar determinations to assess the concentration dependent effects of FGF-2, in the presence of a constant level of 50 ng/ml BMP-2, revealed essentially identical results. For example, the peak dose-response for FGF-2's efficacy was 50 ng/ml, the same as that of BMP-2.

High BMP-2 Induces a Non-Cardiac Phenotype: Non-precardiac mesoderm was explanted from the posterior lateral plate of stage 6 embryos as described in Experimental Procedures and cultivated in the presence of FGF (50 ng/ml) and BMP-2 (25, 50 or 250 ng/ml). Explants were reacted to detect AP activity (histochemistry), presence of sarcomeric α-actin (immunohistochemistry), or expression of the Dlx-5 (RT/PCR) as described in the Experimental Procedures. It was consistently observed that explants treated with 250 ng/ml BMP that did not undergo cardiogenesis formed a multilayer of cells that was solid, in distinction with the hollow vesicle that is indicative of cardiogenesis. Because evaluation by electron micros-copy revealed an expansive extracellular matrix reminiscent of osteogenic tissue, explants were histochemically reacted to detect alkaline phosphatase activity, high levels of which are associated with cells undergoing osteogenesis. It was consistently observed that explants treated with 250 ng/ml BMP-2 which did not undergo cardiogenesis as revealed by absence of beating and sarcomeric α-actin immunostaining exhibited high levels of alkaline phosphatase activity. By contrast, cardiogenic vesicles that formed during treatment with 250 ng/ml BMP-2 were indistinguishable from cardiogenic explants treated with 50 ng/ml BMP-2: alkaline phosphatase was never detected while sarcomeric α-actin was always detected. Alkaline phosphatase activity was never detected in non-cardiogenic explants that had been treated with lower BMP-2 levels. The BMP-inducible homeobox transcription factor Dlx-5, an early osteogenic marker, was up-regulated in BMP concentration-dependent fashion within 30 minutes of BMP+FGF application.

Transient Exposure to BMP and FGF Is Sufficient to Induce Cardiogenesis: It was of interest to consider whether a transient signaling event is sufficient to initiate cardiogenesis, consistent with the notion that migrating mesoderm cells may be only transiently positioned to receive a cardiogenic signal during gastrulation. Therefore, it was determined whether transient exposure of non-precardiac mesoderm to BMP was sufficient to induce the cardiogenic pathway, whereas constant exposure to FGF, in accord with its perceived role as a “survival” molecule, was necessary to maintain specified cells in the cardiogenic pathway.

FIG. 5A shows results from experiments to determine the duration of BMP exposure required to specify cardiogenesis. Cultures were initiated with 50 ng/ml BMP-2 and FGF-2 in Medium 199. At the indicated intervals, BMP-containing medium was exchanged for medium containing only FGF-2 and cultures were continued to the 48 hour endpoint. Explants were evaluated for cardiac differentiation as indicated by formation of a rhythmically contractile vesicle. As expected, cardiogenesis did not occur in the absence of BMP-2 (FIG. 5A, 0 hr exposure). However, exposure for only the first 15 minutes of the 48 hour culture period was sufficient to cause cardiac differentiation in one of six explants, and, only 30 minute treatment induced cardiogenesis in a significant percentage of 13 explants tested. The percentage of cardiogenic explants increased with duration of BMP-2 treatment, with the exception of a consistent decline in explants that were treated for only 2 hours. These experiments demonstrate that brief exposure to BMP-2 at the beginning of the culture period is sufficient to initiate cardiogenesis and that increasing the duration of exposure increases the incidence of contractile explants. In related experiments, it was determined that treatment with BMP-2 at the beginning of the culture period was crucial; explants from which BMP-2 was withheld for the first 24 hours of the culture period did not undergo cardiogenesis.

Reciprocal experiments were performed in which non-precardiac mesoderm was continuously treated with BMP-2 for 48 hour while FGF-2 was applied for variable periods. As shown in FIG. 5B, although cardiogenic differentiation was dependent on treatment with FGF-2, only 15 minutes exposure was sufficient to support differentiation in the majority of explants. Surprisingly, only 30 minutes exposure supported differentiation in 100% of the explants, a finding that has been observed in 40 consecutive repetitions. As in the case of BMP, extending FGF treatment to 2 hour decreased the incidence of contractile explants, followed by increases after longer exposures which however did not approach the 100% incidence of differentiation observed when FGF was limited to the first 30 minutes of the culture period.

Based on the findings in FIG. 5, it was of interest to ascertain the incidence of cardiogenic explants generated by exposure to FGF and BMP for only the first 30 minutes of the culture period. It was observed that such treatment resulted in a cardiogenic incidence of 40%, suggesting, in accord with the data in Fig. SA, that prolonged treatment with BMP is required to attain 100% cardiogenic explants.

FGF and BMP Cooperate to Induce SRF and cNkx-2.5: Serum response factor (SRF), which is involved in the transcription of several cardiac genes including the cardiac and skeletal α-actins and sm22α, is induced by FGF. Parker T G et al., J Biol Chem 267:3343-3350 (1992); Moss J B et al., J Biol Chem 269:12731-12740 (1994). Chicken Nkx-2.5, the homologue of Drosophila tinman, is a transcription factor that is expressed in mesoderm and endoderm cells of the cardiac domain in the embryo; in both Drosophila and avians, tinman/cNkx-2.5 has been shown to be induced by BMP. Schultheiss T M et al., Genes Dev 11:451-462 (1997). SRF and Nkx-2.5 heterodimers strongly up-regulate the transcription of several cardiac genes. Chen C C and R J Schwartz, Mol Cell Biol 16:6372-6384 (1996). Since BMP and FGF cooperatively induce cardiogenesis, it was of interest to determine whether BMP-2 and FGF-2 respectively up-regulate cNkx-2.5 and SRF in non-precardiac mesoderm. Determinations were performed in which explants were treated for 10 or 24 hours with either FGF-2, BMP-2 or both, followed by conventional RT/PCR analysis using primer pairs that amplify SRF, cNkx-2.5 and GAPDH. Thirty-five cycles of PCR amplification, followed by EtBr staining, were performed. In Table 1, “+” indicates detection of EtBr-stained PCR product and “−” indicates that PCR products were not seen. As shown in Table 1, freshly explanted non-cultured posterior mesoderm (0 hr) revealed the presence of GAPDH in all instances, whereas Nkx-2.5 was detected in only 1 of 7 explants and SRF was barely detectable in 3 of 7 explants. Treatment with BMP alone for 10 or 24 hours induced no ethidium bromide-detectable Nkx-2.5 (or SRF) cDNA. Similarly, treatment with FGF alone induced neither transcription factor after 10 hours, although SRF (and Nkx-2.5) was detected in 1 of 4 explants after 24 hours. By contrast, explants treated with both BMP and FGF for 10 or 24 hours exhibited both Nkx-2.5 and SRF in nearly every instance.

TABLE 1

| Growth Factor | Duration | | GAPDH | | Nkx-2.5 | | SRF | |
|---|---|---|---|---|---|---|---|---|
| Added | (hr) | “n” | + | − | + | − | + | − |
| None | 0 | 7 | 6 | 1 | 1 | 6 | 3 | 4 |
| BMP-2 | 10 | 3 | 3 | 0 | 0 | 3 | 0 | 3 |
| FGF-2 | 10 | 3 | 3 | 0 | 0 | 3 | 0 | 3 |
| FGF-2 + BMP-2 | 10 | 3 | 3 | 0 | 3 | 0 | 3 | 0 |
| BMP-2 | 24 | 3 | 2 | 1 | 0 | 3 | 0 | 3 |
| FGF-2 | 24 | 4 | 3 | 1 | 1 | 3 | 1 | 3 |
| FGF-2 + BMP-2 | 24 | 6 | 6 | 0 | 6 | 0 | 5 | 1 |

To more sensitively perform this assessment, as well as to determine whether the cardiac transcription factor GATA-4 was induced, the semi-quantitative PCR determination was performed. Posterolateral non-precardiac mesoderm was explanted from stage 6 embryos and cultured in the presence of 50 ng/ml of either BMP, FGF or both BMP and FGF. RNA from each explant was reverse-transcribed (RT) and one-fourth of each RT product was subjected to PCR amplification in the presence of ³²P-α-dCTP for 28 cycles, during which the accumulation of PCR products was linear. The radioactive PCR products were separated on a 4.5% polyacrylamide gel followed by phosphorimaging and ImageQuant analysis. Normalization of each transcription factor cDNA to the amount of amplified GAPDH cDNA indicates the extent of induction by each growth factor. Explants treated with BMP only did not induce Nkx-2.5 (or SRF or GATA-4). Similarly, explants treated with FGF only did not induce SRF (or Nkx-2.5 or GATA-4). However, explants treated with FGF and BMP induced approximately 7- and 15-fold increases in SRF and Nkx-2.5, respectively, as assessed by ImageQuant analysis. Although GATA-4 was not appreciably amplified after 28 cycles, conventional PCR using 40 cycles revealed the presence of GATA-4 after treatment with BMP and FGF for 24 and 48 hours. An increasing amplification of GAPDH in explants treated with BMP, FGF, and BMP+FGF, reflecting the respective increases in cell proliferation, was also observed.

1. A composition comprising a purified mixture of a bone morphogenetic protein (BMP) and a fibroblast growth factor (FGF) selected from BMP-2 and FGF-2, BMP-4 and FGF-2, or BMP-4 and FGF-4.

2. The composition of claim 1, wherein the molar ratio of the BMP to the FGF is 1:1.

3. The composition of claim 1, wherein the composition additionally comprises a matrix material.

4. The composition of claim 3, wherein the matrix material is collagen.

5. The composition of claim 1, wherein the composition comprises a purified mixture of BMP-2 and FGF-2.

6. The composition of claim 1, wherein the composition comprises a purified mixture of BMP-4 and FGF-2.

7. The composition of claim 1, wherein the composition comprises a purified mixture of BMP-4 and FGF-4.

8. A method for inducing cardiogenesis in cells of non-cardiac lineage, comprising the steps of:
exposing the cells to a purified mixture of a BMP and an FGF selected from BMP-2 and FGF-2, BMP-4 and FGF-2, or BMP-4 and FGF-4; and
observing the development of rhythmic and synchronously contractile cells.

9. The method of Claim8, further comprising the step of:
confirming the development of rhythmic and synchronously contractile cells by measuring the expression of sarcomeric α-actin in the cells.

10. The method of claim 8, wherein both the BMP and the FGF have a concentration from about 5 ng/ml to about 1,000 ng/ml.

11. The method of claim 8, wherein both the BMP and the FGF have a concentration of about 50 ng/ml.

12. The method of claim 8, wherein the exposure to the mixture of BMP and FGF is achieved by exogenously applying a mixture of the proteins to the cells.

13. The method of claim 8, wherein the exposure to the mixture of BMP and FGF is achieved by transforming the cells with a genetic construct encoding the BMP and the FGF.

14. The method of claim 8, wherein the exposure is in vivo.

15. The method of claim 8, wherein the exposure is in vitro.

16. The method of claim 8, wherein the cells are exposed to a purified mixture of BMP-2 and FGF-2.

17. A method for inducing cardiogenesis in cells of non-cardiac lineage, comprising the steps of:
exposing the cells to a purified mixture of a BMP and an FGF selected from BMP-2 and FGF-2, BMP-4 and FGF-2, or BMP-4 and FGF-4; and
measuring the expression of SRF and Nkx-2.5 in the cells.

18. A method for inducing cardiogenesis in cells of non-cardiac lineage, sing the steps of: exposing the cells to a purified mixture of a BMP and an FGF selected from and FGF-2, BMP-4 and FGF-2, or BMP-4 and FGF-4; and continuing to expose the cells to the BMP without the FGF.


SEQUENCE LISTING (14 sequences)

SEQ ID NO: 1 - DNA, 25 bp, Artificial Sequence
  Description of Artificial Sequence oligonucleotide PCR primer
tggaattcggntggvangaytggat

SEQ ID NO: 2 - DNA, 25 bp, Artificial Sequence
  Description of Artificial Sequence oligonucleotide PCR primer
gaggatccggnacrcarcangcytt

SEQ ID NO: 3 - DNA, 20 bp, Artificial Sequence
  Description of Artificial Sequence oligonucleotide PCR primer
acgccatcactatcttccag

SEQ ID NO: 4 - DNA, 20 bp, Artificial Sequence
  Description of Artificial Sequence oligonucleotide PCR primer
cagccttcactaccctcttg

SEQ ID NO: 5 - DNA, 20 bp, Artificial Sequence
  Description of Artificial Sequence oligonucleotide PCR primer
ctacgaactggagagaaggt

SEQ ID NO: 6 - DNA, 20 bp, Artificial Sequence
  Description of Artificial Sequence oligonucleotide PCR primer
gtaggcgttgtagctatagg

SEQ ID NO: 7 - DNA, 20 bp, Artificial Sequence
  Description of Artificial Sequence oligonucleotide PCR primer
cagcaactctctcgtacgga

SEQ ID NO: 8 - DNA, 20 bp, Artificial Sequence
  Description of Artificial Sequence oligonucleotide PCR primer
tctgcaggacagctccaggt

SEQ ID NO: 9 - DNA, 20 bp, Artificial Sequence
  Description of Artificial Sequence oligonucleotide PCR primer
ctcctactccagcccttacc

SEQ ID NO: 10 - DNA, 20 bp, Artificial Sequence
  Description of Artificial Sequence oligonucleotide PCR primer
gccctgtgccatctctcctc

SEQ ID NO: 11 - DNA, 19 bp, Artificial Sequence
  Description of Artificial Sequence oligonucleotide PCR primer
gctccgccggcacctaccc

SEQ ID NO: 12 - DNA, 20 bp, Artificial Sequence
  Description of Artificial Sequence oligonucleotide PCR primer
ggagcgcgacgagccctgag

SEQ ID NO: 13 - DNA, 20 bp, Artificial Sequence
  Description of Artificial Sequence oligonucleotide PCR primer
ggcgactcttgatgagaaca

SEQ ID NO: 14 - DNA, 20 bp, Artificial Sequence
  Description of Artificial Sequence oligonucleotide PCR primer
gcttccagctcctcttccag